(19) United States
(12) Patent Application Publication
(10) Pub. No.: US 2026/0041496 A1
(43) Pub. Date: Feb. 12, 2026

(54) METHOD AND MEDICAL SYSTEM FOR ADJUSTING A REFERENCE X-RAY IMAGE

(71) Applicant: Siemens Healthineers AG, Forchheim (DE)

(72) Inventor: Marcus Pfister, Bubenreuth (DE)

(21) Appl. No.: 19/285,808

(22) Filed: Jul. 30, 2025

(30) Foreign Application Priority Data
Aug. 8, 2024 (DE) 10 2024 207 539.6

(51) Int. Cl. A61B 34/20; A61B 6/00; A61B 6/12; A61B 34/30; A61B 90/00; A61M 25/01

(52) U.S. Cl. CPC A61B 34/20; A61B 6/12; A61B 6/5235; A61B 2034/2061; A61B 2034/303; A61B 2090/065; A61M 2025/0166

(57) ABSTRACT

A method for adjusting a reference X-ray image includes: providing the reference X-ray image of a hollow organ; providing a segmentation of an original profile of the hollow organ in the reference X-ray image; recording at least one live X-ray image, registered with the reference X-ray image, of the object introduced into the hollow organ; segmenting the object in the live X-ray image; carrying out at least one sensor measurement of the optical fiber by the shape-acquisition system; evaluating the sensor measurement(s) with regard to at least one item of haptic information in respect of the object; setting boundary conditions for the current profile of at least part of the hollow organ using the position of the object and the evaluated item of haptic information; and adjusting and/or deforming the reference image using the boundary conditions such that a current profile of the hollow organ is modeled/reproduced.

The present patent document claims the benefit of German Patent Application No. 10 2024 207 539.6, filed Aug. 8, 2024, which is hereby incorporated by reference in its entirety.

### TECHNICAL FIELD

The disclosure relates to a method for adjusting a reference X-ray image of a hollow organ of a patient to a live X-ray image as well as to a medical system for carrying out a method of this kind.

### BACKGROUND

In minimally invasive examinations or interventions, (e.g., supported by C-arm angiography systems), therapies and/or diagnoses are carried out with the aid of instruments introduced into the body via small incisions, for example, in the groin. As a rule, radioscopy (in particular fluoroscopy) has previously been used to navigate to the region of interest, (e.g., a vascular target), or to visualize catheters and other instruments. In order to minimize the drawback of the radiation, more recent methods use a 3D fiber optic, which may identify the shape and position of an introduced optical fiber by intrinsic light reflections. Thus, for example, guide wires in a body may be visualized by measuring their shape and position and virtually representing them in a model or an image recording of the body. For example, treatment of an aortic aneurysms by inserting a stent graft is a specific therapy in which these instruments are of use. The introduced instrument, for example a guide wire or a catheter, has for this purpose an optical fiber over its length for localization, which fiber is permanently connected to the instrument. Multifunctional shape-sensing fibers and their functions are known, for example, in the framework of the Pathfinder technology (The Shape-Sensing Company), (see, e.g., shapesensing. com/pathfinder-platform and shapesensing. com).

As depicted in FIG. 2, for example, minimally invasive procedures also benefit from reference X-ray images 22 (for example, pre-op X-ray images in 2D or 3D) and live X-ray images 23 (for example fluoroscopy images in 2D) being registered and overlaid or being jointly visualized with the fiber optic. However, the problem lies in the reference X-ray image showing the structure of the hollow organ at an instant before the instrument has been introduced. After introduction of the (rigid) instrument into the hollow organ, the anatomy thereof deforms and the overlaid images no longer coincide, as may be seen in FIG. 2. This results in uncertainties about the actual anatomy at the current instant, complications may occur and the position of the instrument in the hollow organ is no longer clear. In the worst case the intervention has to be terminated.

Methods exist that take into account the deviation on the basis of the live X-ray image and deform the reference X-ray image in accordance with the live X-ray image, (see, e.g., DE 10 2010 012621 A1). However, these methods are frequently not accurate enough owing to the lack of anatomical items of information in the live X-ray image.

### SUMMARY AND DESCRIPTION

It is the object to provide a method that improves the quality of the representation of the current profile of the hollow organ and to provide a medical system suitable for carrying out the method.

The object is achieved by a method for adjusting a reference X-ray image of a hollow organ of a patient to a live X-ray image and by a medical system as disclosed herein. The scope of the present disclosure is defined solely by the appended claims and is not affected to any degree by the statements within this summary. The present embodiments may obviate one or more of the drawbacks or limitations in the related art.

The method for overlaying a reference X-ray image of a hollow organ of a patient with a live X-ray image of an object introduced into the hollow organ, wherein the object is structurally connected to an optical fiber, (e.g., multi-functional shape-sensing (MFSS) fiber), which is part of a fiber optic shape-acquisition system, includes providing the reference X-ray image of the hollow organ. The method further includes providing a segmentation of the original profile of the hollow organ in the reference X-ray image. The method further includes recording at least one live X-ray image, registered with the reference X-ray image, of the object introduced into the hollow organ. The method further includes segmenting the object in the live X-ray image. The method further includes carrying out at least one sensor measurement of the optical fiber by the shape-acquisition system, in particular at the same time as recording. The method further includes evaluating the sensor measurement(s) with regard to at least one item of haptic information in respect of the object, in particular a friction force and/or contact force (i.e., wall contact force) that acts on the object. The method further includes selecting boundary conditions for the current profile of at least part of the hollow organ using the position of the object and the evaluated item of haptic information, in particular friction force and/or contact force. The method further includes adjusting and/or deforming the reference image, starting from the segmented original profile of the hollow organ, using the boundary conditions in such a way that a current profile of the hollow organ is modeled/reproduced. The method may provide improved adjustment of the reference image to the current actual profile of the hollow organ and therewith a more accurate representation due to the additional items of information.

Instead of static measured values, like the position of the object, dynamic effects, (e.g., forces that act on the object and transferred therewith also on the hollow organ or a hollow organ wall), are also introduced in the adjustment of the current profile of the hollow organ, whereby the adjustment process is optimized. This results in improved planning and safer implementation of an interventional procedure. The care of the patient is improved by the more exact representation of the hollow organ; complications may be avoided. The use of shape sensing technology requires only low structural outlay but achieves good results by comparison. In particular, the object is formed by a catheter apparatus and the optical fiber is structurally connected to the catheter apparatus. The reference image is recorded, for example, by a 3D pre-op X-ray image (i.e., an X-ray image recorded before the op), for example, by computed tomography (CT) or cone beam CT (CBCT). The live X-ray image may be formed by a 2D fluoroscopy image.

The measuring apparatus has a fiber optic shape-acquisition system and at least one multi-functional shape-sensing fiber. A fiber optic shape acquisition allows, for example, for the curvature and shape of an optical fiber to be determined in 2D and 3D. A fiber optic shape-acquisition system, as may also be used in the method, may include a sensor, a measuring device, and a computing unit (evaluation unit) with algorithms for evaluating the measured data. When measuring, the strain in the fiber is ascertained by interference of a plurality of light beams that are transmitted through the fiber and are reflected in the fiber. With a fiber made of a bundle of a plurality of fiber-optic conductors, some of the outer fiber-optic conductors experience a relative stress or compression relative to the central fiber-optic conductor and therefore register positive or negative induced changes in strain. The relative strains of the fibers are measured and processed to calculate a local curvature or the bending radius. To determine the curvature profile of the fibers, the measurements are processed with specific reconstruction algorithms. Radii of curvature, directions of curvature, pressure, temperature, various forces, and various torsions may also be ascertained in addition to curves around a point.

According to one embodiment, the evaluation ascertains a friction force that acts on the object and reproduces the current profile using the position of the object and the evaluated friction force. Known shape-acquisition systems are capable of ascertaining friction forces along the optical fiber. This information may be used for the modeling. The hollow organ or the hollow organ walls are assumed as the surroundings of the object, which experience the force. In addition to the modeling along the position of the object, (e.g., catheter), the hollow organ may thus be adjusted to the friction forces. In particular, a direction of the friction force is ascertained for this purpose and used in such a way that the original profile of the hollow organ and its entry points of the hollow organ are distorted/shaped in the same direction. The hollow organ is therefore adjusted not only to the shape, but also to the direction of the force and potentially intensity of the friction force, e.g., in a manner that the hollow organ is pulled in the corresponding direction like a flexible hose. This may also include the exit points of the hollow organ, which are pulled in the direction.

According to a further embodiment, the evaluation ascertains a contact force that acts on the object and reproduces the current profile using the position of the object and the evaluated contact force. Known shape-acquisition systems are likewise capable of ascertaining contact forces along the optical fiber. The hollow organ or the hollow organ walls are assumed as the surroundings of the object, which experience the contact force. Advantageously, the current profile of the hollow organ is reproduced in such a way that at regions with a contact force below a threshold value, the hollow organ wall has a spacing from the object. The contact force below the threshold value, (e.g., a previously selected or automatically set threshold value), is therefore interpreted in such a way that there is no contact with the hollow organ wall. The spacing may be selected or automatically set accordingly. Even further sensor measurements may be used for this, for example, also a curve of the optical fiber.

According to a further embodiment, the sensor measurement(s) are evaluated with regard to further items of information and the further items of information are used to reproduce the current profile. These items of information may be the bending radius, further forces, pressure, or temperature.

According to a further embodiment, the current positions of entry points of the hollow organ are reproduced using the item of haptic information. These positions, like the profile of the hollow organ, may be shifted, distorted, or rotated in accordance with the measured forces in order to achieve a realistic representation of the current hollow organ.

According to a further embodiment, a series of at least two live X-ray images registered with the reference X-ray image is recorded and segmented in chronological order and sensor measurements in each case recorded at the same time as the live X-ray images are evaluated with regard to an item of haptic information and with regard to a change over time in the item of haptic information, and the change over time in the item of haptic information is additionally used for the reproduction of the respective current profile. The use of changes over time makes an even more exact adjustment of the current profile of the hollow organ possible, in particular an incremental change, so the respective starting points may also be included.

The disclosure also includes a medical system for carrying out the method, having an X-ray device for recording a live X-ray image, a provision unit for providing a reference image, a catheter apparatus with a catheter, a measuring apparatus having a multi-functional shape-sensing fiber arranged on the catheter apparatus, a measuring unit and an evaluation unit for evaluation of the sensor measurements of the optical fiber in such a way that an item of haptic information is determined, a calculation unit, a system controller for controlling the method, and an image processing unit for adjusting and/or deforming the reference image, starting from the segmented original profile of the hollow organ, using the boundary conditions in such a way that a current profile of the hollow organ is modeled/reproduced. In particular, the medical system also includes a robotic control system with a driving apparatus and a control unit for moving and actuating the robotically controllable catheter apparatus through a hollow organ of a patient.

### BRIEF DESCRIPTION OF THE DRAWINGS

The disclosure is explained in more detail in the drawings below on the basis of schematically represented exemplary embodiments without limiting the disclosure to these exemplary embodiments.

FIG. 1 depicts a sequence of acts of a method for adjusting a reference image to a live X-ray image, according to an example.

FIG. 2 depicts a view of a reference image, a live X-ray image and the overlaying thereof, according to an example.

FIG. 3 depicts a view of a catheter with indicated friction force, according to an example.

FIG. 4 depicts a view of a current profile of the hollow organ deformed according to FIG. 3, according to an example.

FIG. 5 depicts a view of a catheter with indicated contact force, according to an example.

FIG. 6 depicts a view of a current profile of the hollow organ deformed according to FIG. 5, according to an example.

FIG. 7 depicts a further view of a catheter with indicated friction force, according to an example.

FIG. 8 depicts the friction force plotted against the catheter according to FIG. 7, according to an example.

FIG. 9 depicts a further view of a catheter with indicated contact force, according to an example.

FIG. 10 depicts the contact force plotted against the catheter according to FIG. 9, according to an example.

FIG. 11 depicts a view of the progression of a catheter over time, according to an example.

FIG. 12 depicts a medical system for carrying out the method, according to an example.

### DETAILED DESCRIPTION

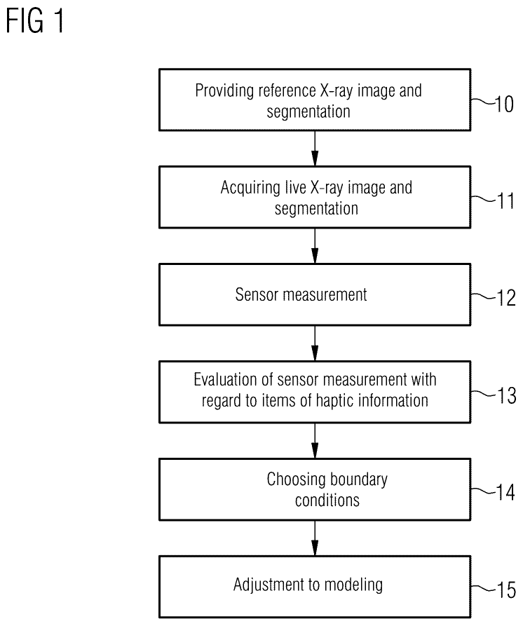

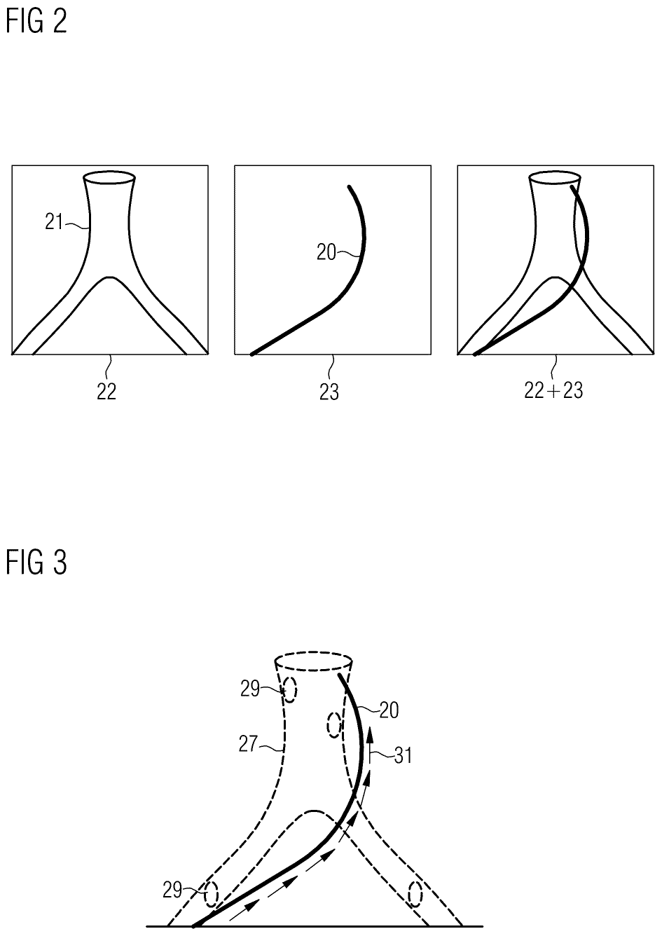

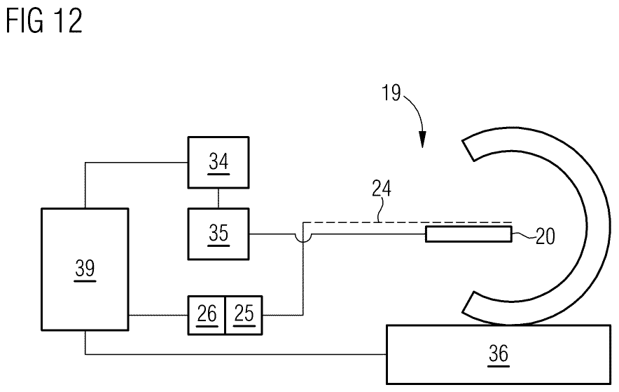

FIG. 1 shows acts of a method for adjusting a reference image to a live X-ray image. FIG. 12 shows the associated medical system for carrying out the method, controlled by a system controller 39. For an interventional procedure, in which an object, such as a catheter 20 or a catheter apparatus 19, is moved through a hollow organ 21 with X-ray monitoring, at least one reference image 22 (such as a 3D pre-op X-ray image) is used for planning. The reference image was recorded, for example, with contrast agent (DSA=digital subtraction angiography), so an exact mapping of the hollow organ 21 (for example, a blood vessel or a blood vessel system including two or more blood vessels with cardiac openings) exists. The hollow organ 21 deforms following introduction of the (e.g., rigid) object. During the movement of the object through an X-ray device 36, recorded live X-ray images 23 (for example, 2D fluoroscopy images) may show the object clearly, but not the hollow organ 21 or show the hollow organ 21 only indistinctly, so it is not possible to identify how the current profile 28 of the hollow organ 21 actually is now. As may be seen in FIG. 2, the original profile 27 of the hollow organ 21 no longer matches the current profile 27, although this is indicated in the live X-ray image only by the object. For the sake of simplicity, the reference image 22 is shown two-dimensionally, but is frequently three-dimensional. The live X-ray image may then be overlaid in the corresponding plane. The method evaluates dynamic items of information in addition to static items of information by way of positions of the object, and this dynamic information is used for adjusting the reference image to the current profile of the hollow organ in order to thus obtain an exact and realistic update of the profile of the hollow organ.

At least one or more multi-functional shape-sensing fibers 24 are arranged on the catheter apparatus 19, for example, partially or completely along the catheter 20 or guide wire, and these fibers are part of a fiber optic shape-acquisition system. The fiber optic shape-acquisition system also has a measuring device 25 and an evaluation unit 26 with algorithms for evaluating the measured data. The fiber 24 may be permanently connected to the catheter apparatus 19, for example, in the region of the catheter 20. The shape-acquisition system measures a strain in the fiber by interference of a plurality of light beams that are transmitted through the fiber and reflected in the fiber. With a fiber composed of a bundle of a plurality of fiber-optic conductors, some of the outer fiber-optic conductors experience a relative stress or compression in relation to the central fiber-optic conductor and therefore register positive or negative induced changes in strain. The relative strains of the fibers are measured and processed to calculate a local curvature or the bending radius. In order to determine the curvature profile of the fiber, the measurements are processed with specific reconstruction algorithms. Radii of curvature, directions of curvature, pressure, temperature, various forces (for example, friction force and wall force), and torsions may be measured or evaluated from the measurements, in addition to curves around a point.

In act 10, a reference X-ray image of the hollow organ is provided, wherein the original profile of the hollow organ (for example, the hollow organ contours) is segmented or will be segmented in the reference X-ray image. The reference X-ray image may be a 3D pre-op X-ray image recorded before an intervention. The reference X-ray image may have been recorded, for example, by an angiography X-ray device or a CT with a flow of contrast agent through the hollow organ. The reference X-ray image may be taken, for example, from a memory or a database or be recorded directly before the intervention. The reference X-ray image may be provided at the beginning of the interventional procedure or before the intervention.

The object, for example, the catheter apparatus 19, is already located in the hollow organ at the instant of act 11. In certain examinations, the robotically driven catheter apparatus 19 may be moved by a robotic drive 33 and a robot controller 34 through the hollow organ to a desired location, wherein a guide wire may optionally be present. The object may also have been moved manually by an experienced operator.

In act 11, a live X-ray image 23 is recorded in which the object located in the hollow organ is mapped, although the hollow organ itself frequently cannot be seen clearly or cannot be seen at all. The live X-ray image may be formed by a fluoroscopy image and may be a projection image (two-dimensional), in order to save on the dose. The reference image (or the representation thereof in the plane of the live X-ray image) and the live X-ray image may cover congruent regions, but may also merely overlap in parts. In certain examples, the live X-ray image covers a smaller region. A segmentation is likewise carried out with the live X-ray image, primarily the object is segmented here. For example, the position of the object is then determined from this. The live X-ray image may be recorded, for example, by way of an X-ray device 36, e.g., an angiography X-ray device with a C-arm, on which an X-ray detector and an X-ray source are arranged.

In act 12, at least one sensor measurement or also a plurality of sensor measurements of the optical fiber 24 is carried out by a measuring device 25 of the shape-acquisition system at the same time as recording the live X-ray image. The implementation of such sensor measurements is known and, for example, described above. For example, a plurality of light beams is transmitted through the fibers and reflected in the fibers and the corresponding interference then recorded that is a function of positively or negatively induced changes in the strain of the fiber.

The optical shape-acquisition system may be registered with the reference image and/or the X-ray device 36 generating the live X-ray image. All positions relative to one another are also then determined hereby.

In act 13, the sensor measurements are evaluated with regard to at least one item of haptic information in respect of the object. The evaluation is carried out, for example, by an evaluation unit 26 of the optical shape-acquisition system in a manner known from the prior art. Thus, for example, the required value is ascertained form the changes in strain, for example, the friction force and/or the contact force (e.g., wall contact force), which acts on the object. The values may be ascertained, for example, for each position along the object if the optical fiber is appropriately arranged on the object. It may accordingly be assumed that the ascertained friction force and/or the contact force come about due to an interaction between the hollow organ, or a hollow organ wall, and the object. Further values may also be ascertained from the sensor measurements, for example a curve or a temperature.

In act 14, appropriate boundary conditions are subsequently selected that are used to generate the current profile of the hollow organ from the original profile. The position of the object segmented from the live X-ray image and evaluated items of haptic information are used as the boundary conditions. First, a first boundary condition is that the object is located completely inside the hollow organ with the current profile of the hollow organ. The position may either be taken from the segmentation, the evaluation of the optical shape-acquisition system, or another position measurement (for example, EM tracking). A calculation unit (not shown), which may be part of the system controller, may be used for this.

In the case of a contact force, it may be assumed that the force is produced by the contact of the object with a hollow organ wall. If the contact force has a high value in a specific region, it is assumed that there is close contact between the object and the hollow organ wall here. If the contact force is very low, for example, below a specific value, it is assumed that there is little or no contact with the hollow organ wall or even a spacing from the hollow organ wall. The corresponding boundary conditions are therefore, for example, a direct contact of the object with the inner hollow organ wall or a spacing from the inner hollow organ wall.

In the case where a friction force occurs, it may be assumed that the object is moved through the hollow organ and the friction force comes about as a result. The same friction force as on the object therefore also acts on the hollow organ wall. The direction of the friction force is an indication of whether the object is being pulled or pushed. As a result, the hollow organ is deformed to a greater or lesser extent relative to the direction and relative to the intensity of the friction force. The boundary conditions are then the corresponding distortions in corresponding intensity. These assumptions now constitute the boundary conditions for the current profile of the hollow organ and, for example, also the current position of the exit points of the hollow organ.

Furthermore, boundary conditions may include that regions of the hollow organ more distant from the object are not deformed and instead correspond to the original profile. It may also be a boundary condition that a smooth transition may exist between the portion of the hollow organ in which the object is located and the other (undeformed) portions of the hollow organ. A boundary condition may also be that specific lengths of sections of the hollow organ do not change, be it in the plane (2D) or in 3D. It may also be a boundary condition that the diameter of the hollow organ does not change or only changes within specific limits.

In act 15, the reference image is subsequently deformed, starting from the segmented original profile of the hollow organ, using the boundary conditions, so a current profile of the hollow organ is modeled or adjusted. This is shown, for example, in FIGS. 3 to 6. This may be carried out by an image processing unit (not shown). For the sake of simplicity, in the figures, a 2D projection of the hollow organ/reference image is shown in the plane of the live X-ray image. In certain examples, the deformation takes place in 3D, so a more complex deformation is performed and foreshortening and other problems are also incorporated. Two or more live X-ray images may also be recorded and segmented from different recording directions. Thus, further positions of the object in 3D may be used, which are taken into account in the deformation.

FIG. 3 shows the original profile 27 of the hollow organ and the original positions 29 of the exit points of the hollow organ as well as the catheter 20 in the case of a 2D overlay of reference image and live X-ray image. The arrows 31 symbolize the direction of the friction force, and the length thereof the intensity of the friction force, and these are measured by the optical shape-acquisition system along the catheter 20.

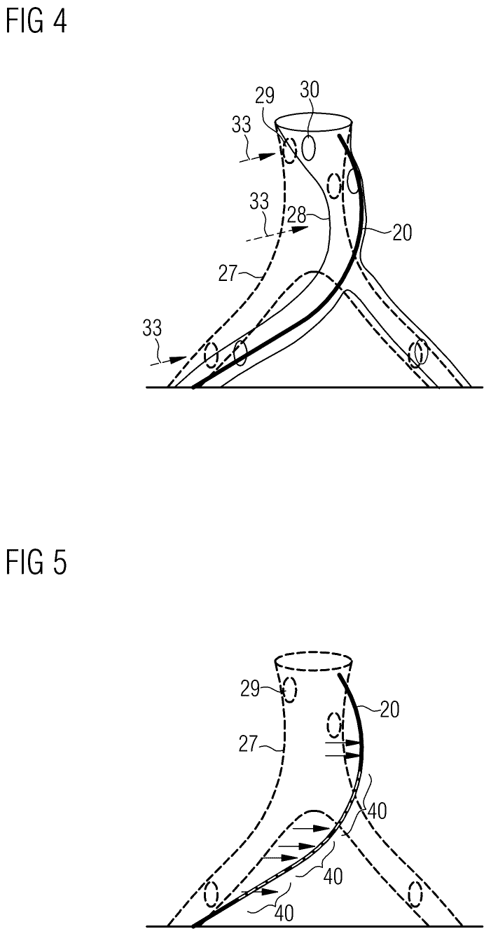

FIG. 4 then shows the end result of the deformation, the current profile 28 of the hollow organ, and the current position 30 of the exit points of the hollow organ in solid lines, with the original contours being shown in broken lines. The position of the catheter 20 as well as the friction force indicated in FIG. 3 were used as boundary conditions for this. The broken-line arrows show the direction of the deformation (in the plane).

FIG. 5 shows the original profile 27 of the hollow organ and the original positions 29 of the exit points of the hollow organ as well as the catheter 20 in the case of a 2D overlay of reference image and live X-ray image. Broken-line regions 40 are shown on the catheter 20, at which regions a contact force below a fixed threshold value was measured. For these broken-line regions 40, it is assumed that there is no contact between the catheter 20 and the inner hollow organ wall.

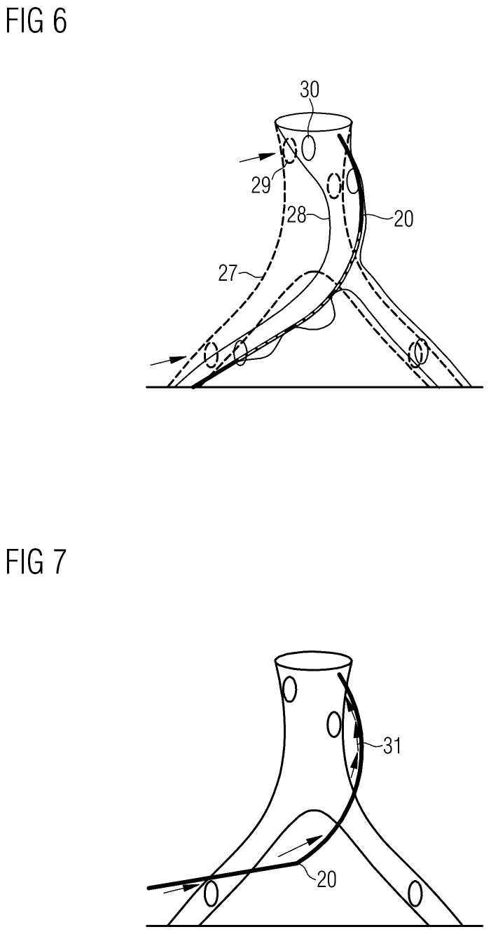

Accordingly, the result of the deformation in FIG. 6 is that the current profile 28 of the hollow organ (or the inner hollow organ wall) in the hatched regions 40 is spaced apart from the catheter 20.

With the aid of various arrows 31, FIG. 7 shows a friction force that changes in direction and intensity over the length of the catheter 20.

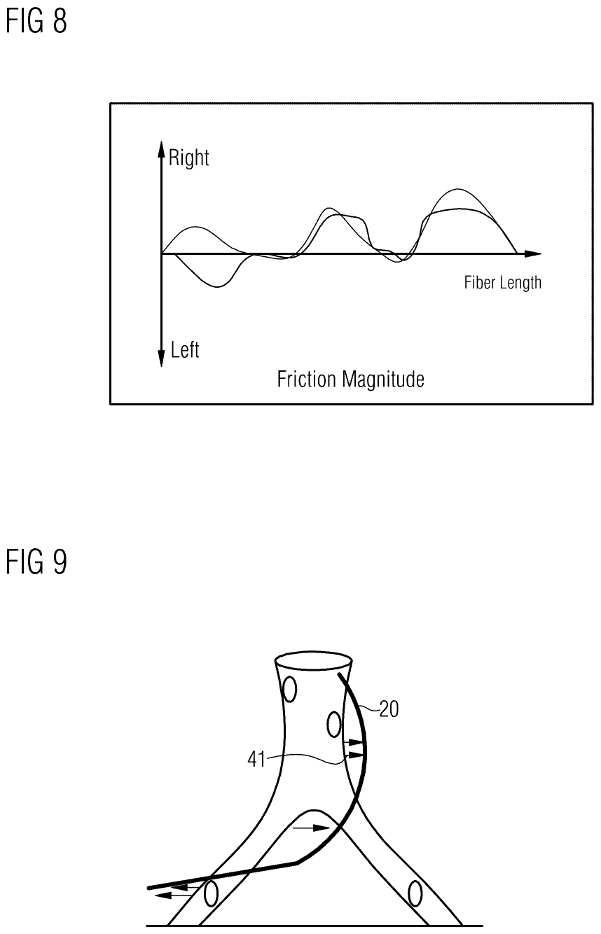

In FIG. 8, the same friction force is plotted against the length of the catheter (x-axis). These may likewise be used as boundary conditions in order to refine the deformation of the original profile to the current profile.

FIG. 9 also shows the direction and intensity of the contact force with the aid of broken-line arrows 41.

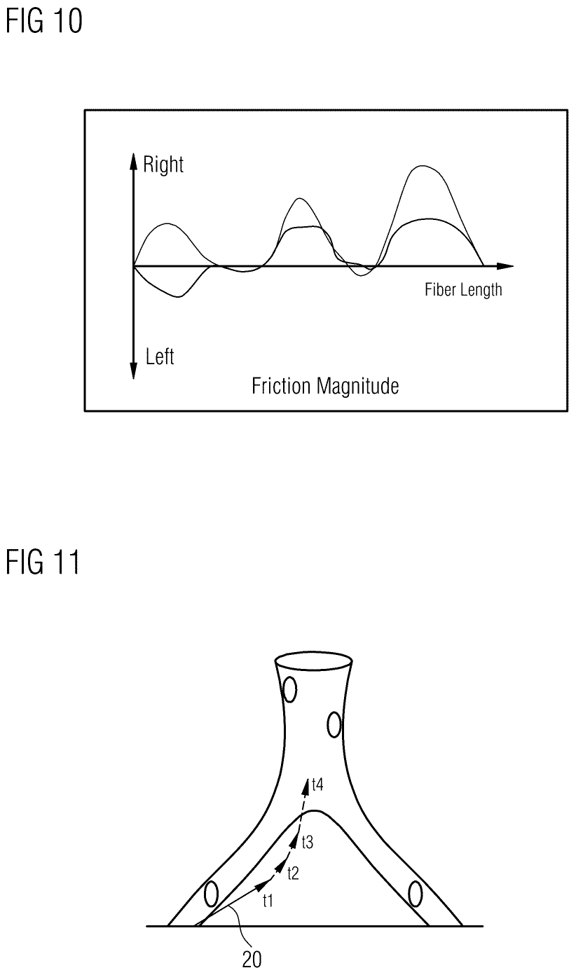

In FIG. 10, the same contact force is plotted against the length of the catheter (x-axis). These may likewise be used as boundary conditions in order to refine the deformation of the original profile to the current profile.

Furthermore, measurements over a change over time, for example of the contact force and/or the friction force, may also be used to create boundary conditions therefrom which the deformation has to satisfy. Thus, a change in the friction force measured over time, for example in successive intervals during the progression of a movement of the object through the hollow organ, may be ascertained. The deformation is then performed, for example, by reproducing this change over time, e.g., the original profile of the hollow organ are distorted with changing direction and intensity, while it is adjusted to the position of the object.

FIG. 11 shows, by way of example, the positions of the tip of the catheter 20 at different instants t1 to t4. The changes over time in the position may also be taken into account in the (for example final) adjustment of the hollow organ.

Overall, the method takes into account not just static (like the position) but primarily dynamic influencing factors in order to deform and model the original profile of the hollow organ, which is known from the reference image, to a current profile. This is based on the knowledge that it is not just the position itself but also the manner in which the position of the object came about which influences the hollow organ and the exit points of the hollow organ.

The method itself may be started or triggered, for example, automatically or by a user. This may be preceded, for example, by a detection of a deviation between the live X-ray image and the object, likewise either manually or automatically.

The method may be used for hollow organs, such as blood vessels and lung vessels, and also hollow organs.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for adjusting a reference X-ray image of a hollow organ of a patient to a live X-ray image of an object introduced into the hollow organ, wherein the object is structurally connected to an optical fiber that is part of a fiber optic shape-acquisition system, the method comprising:
providing the reference X-ray image of the hollow organ;
providing a segmentation of an original profile of the hollow organ in the reference X-ray image;
recording at least one live X-ray image, registered with the reference X-ray image, of the object introduced into the hollow organ;
segmenting the object in the live X-ray image;
carrying out at least one sensor measurement of the optical fiber by the fiber optic shape-acquisition system;
evaluating the at least one sensor measurement with regard to at least one item of haptic information in respect of the object;
selecting boundary conditions for a current profile of at least part of the hollow organ using a position of the object and the at least one item of haptic information; and
adjusting and/or deforming the reference image, starting from the segmented original profile of the hollow organ, using the boundary conditions in such a way that the current profile of the hollow organ is modeled or reproduced.

2. The method of claim 1, wherein the object is formed by a catheter apparatus and the optical fiber is structurally connected to the catheter apparatus.

3. The method of claim 1, wherein the at least one item of haptic information is a friction force, and
wherein the current profile is reproduced using a position of the object and the friction force.

4. The method of claim 3, wherein a direction of the friction force is ascertained and used in such a way that the original profile of the hollow organ and entry points of the hollow organ are distorted or shaped in a same direction.

5. The method of claim 1, wherein the at least one item of haptic information is a a contact force, and
wherein the current profile is reproduced using the position of the object and the contact force.

6. The method of claim 5, wherein the current profile is reproduced in such a way that a wall of the hollow organ has a spacing from the object at regions with a contact force below a threshold value.

7. The method of claim 1, wherein the at least one sensor measurement is evaluated with regard to a further item of information, and
wherein the further item of information is used to reproduce the current profile.

8. The method of claim 7, wherein the further item of information comprises a bending radius.

9. The method of claim 1, wherein current positions of entry points of the hollow organ are reproduced using the at least one item of haptic information.

10. The method of claim 1, wherein a series of at least two live X-ray images registered with the reference X-ray image are recorded and segmented in chronological order,
wherein sensor measurements in each case recorded at a same time as the live X-ray images are evaluated with regard to an item of haptic information of the at least one item of haptic information and with regard to a change over time in the item of haptic information, and
wherein the change over time in the item of haptic information is additionally used for the reproduction of the current profile in each case.

11. The method of claim 1, wherein the optical fiber is a multi-functional shape-sensing fiber.

12. The method of claim 1, wherein the carrying out at least one sensor measurement is performed at a same time as the recording of the at least one live X-ray image.

13. The method of claim 1, wherein the at least one item of haptic information is a friction force and/or a contact force that acts on the object.

14. A medical system comprising:
a catheter apparatus having a catheter;
a provision unit configured to provide a reference image of a hollow organ of a patient and a segmentation of an original profile of the hollow organ in the reference X-ray image;
an X-ray device configured to record a live X-ray image of the catheter apparatus introduced into the hollow organ of the patient, wherein the catheter apparatus is structurally connected to a multi-functional shape-sensing that is part of a fiber optic shape-acquisition system;
a measuring apparatus having the multi-functional shape-sensing fiber arranged on the catheter apparatus, a measuring unit, and an evaluation unit configured to evaluate at least one sensor measurement of the multi-functional shape-sensing fiber in such a way that an item of haptic information is determined;
a controller configured to:
segment the catheter apparatus in the live X-ray image;
carry out at least one sensor measurement of the optical fiber by the fiber optic shape-acquisition system; and
select boundary conditions for a current profile of at least part of the hollow organ using a position of the catheter apparatus and the item of haptic information; and
an image processing unit configured to adjust and/or deform the reference image, starting from the segmented original profile of the hollow organ, using the boundary conditions in such a way that the current profile of the hollow organ is modeled or reproduced.

15. The medical system of claim 14, further comprising:
a robotic control system with a driving apparatus and a control unit configured to move and control the catheter apparatus through the hollow organ of the patient.
